(19) United States
(12) Patent Application Publication
(10) Pub. No.: US 2024/0116017 A1
(43) Pub. Date: Apr. 11, 2024

(54) COMPARTMENTALIZED ARRAYS OF LINKER MOLECULES

(71) Applicant: University of Guelph, Guelph (CA)

(72) Inventors: Huiyan Li, Guelph (CA); Roshan Aggarwal, Guelph (CA)

(21) Appl. No.: 18/376,963

(22) Filed: Oct. 5, 2023

Related U.S. Application Data
(60) Provisional application No. 63/413,754, filed on Oct. 6, 2022.

(51) Int. Cl. B01J 19/00

(52) U.S. Cl. CPC B01J 19/0093; B01J 2219/00612; B01J 2219/00626; B01J 2219/00637; B01J 2219/00659

(57) ABSTRACT

The present application describes compartmentalized arrays of printed linker molecules with physical barriers on the surface, the physical barriers forming one or more compartments surrounding and separating at least a portion of the plurality of distinct regions. The present application also describes a method of fabrication and uses of the compartmentalized arrays.

### CROSS-REFERENCE TO RELATED APPLICATIONS

The present application claims the benefit of priority from co-pending U.S. provisional patent application No. 63/413,754 filed on Oct. 6, 2022, the contents of which are incorporated herein by reference in their entirety.

### FIELD

The present application relates to compartmentalized arrays of printed linker molecules, methods of fabrication and uses thereof.

### BACKGROUND

In the human body, billions of biomolecules and trillions of cells form complex networks to perform various biofunctions. For deciphering the complexity of biological systems, multiplexed bioassays are desired, with microarray technology introduced in the 1980s. DNA/RNA microarrays have seen less use in recent years due to the availability of sequencing technologies, while protein, cell, and extracellular vesicle (EV) microarrays are under active study as multiplexing techniques. Conventional microarrays include a substrate, (e.g. a glass slide) coated with a continuous layer of linker molecules. Bioreagents (e.g., antibodies, cells, EVs) are then printed in (sub)nanoliter spots and bound onto the substrate via the linker molecules. However conventional multiplexed bioanalysis typically employs conventional assays, e.g., 96-well plate-based ELISA, which are single-plex (i.e., measuring one analyte per plate), thus utilizing multiple plates for multiple analytes, and high bioreagent and sample consumption. The slow adoption of conventional microarray techniques is largely due to the challenges specified below.

Compared to the 96-well ELISA, conventional microarray technologies suffer from impaired sensitivity due to lower activity of the printed bioreagents and higher background noise. A contact pin-spotter prints bioreagents by contacting the pins with a glass slide, but presses delicate biomolecules and cells onto the substrate surface, which risks damaging both the bioreagents and the linker layer. Non-contact inkjet spotting techniques have been developed, however bioreagents (especially intact cells) may sediment, aggregate, or experience shear stresses within or when being deposited by the inkjet nozzles. Furthermore, hygroscopic substances are typically added to bioreagent printing solutions to prevent the bioreagents from drying, which may alter the physiological activities of the bioreagents. Each bioreagent typically requires optimization of the printing solution to achieve acceptable printing results. 96-well ELISA systems read signals from the bottom of each well (i.e., assay area), which reduces background noise. In contrast, conventional microarrays typically suffer from higher background noise because the areas surrounding each assay spot are also coated with linker molecules that can bind bioreagents to produce signals. A blocking step is typically employed to reduce background signals, but this blocking step is often inefficient and requires extensive optimization, and may also further degrade bioreagent sensitivity.

Compared to the 96-well ELISA, conventional microarray technologies also suffer from limitations in reproducibility. Lack of reproducibility has been a major challenge in conventional microarray technologies, compared to ELISA. Excess unbound bioreagents in microarray spots can result in tailing and smearing effects upon washing, due to their binding onto the background linkers, affecting data extraction. Inconsistencies during bioreagent printing, e.g. drying and sedimentation of large biomolecules and cells in the spotter pins, may cause changes to the printing solution concentration or viscosity, causing spot-to-spot variations. For contact printing, insufficient rinsing of the pins after each spotting round may result in carryover of the bioreagent. Inhomogeneities in the linker molecules layer may also affect assay reproducibility.

Conventional microarrays typically have reduced accessibility and flexibility. Fabricating conventional microarray-based bioassays requires expensive and complex microarray spotters. Pre-printed antibody microarray chips are commercially available for vendor-selected analytes, but they allow little to no flexibility for end-users to choose analytes based on their needs. Overall, conventional microarray technologies have been developed for decades, however their performance in multiplexed bioassays are still limited due to their limitations in assay sensitivity, reproducibility, and accessibility.

Recently, nanoarrays have been developed for further miniaturization, higher assay sensitivity, and less sample/reagent consumption. However, the fabrication of nanoarrays with biomolecules has typically required complicated nanofabrication skills and instruments, limiting their broader applications.

### SUMMARY

The present application discloses compartmentalized arrays of printed linker molecules and methods of using the arrays for conducting multiplexed bioanalysis. The present application also discloses the methods of fabrication of the arrays.

Accordingly, the present application includes a compartmentalized array of linker molecules comprising:
a) linker molecules printed on a surface of a substrate in a plurality of distinct regions to form a printed array of linker molecules;
b) physical barriers on the surface, the physical barriers forming one or more compartments surrounding and separating at least a portion of the plurality of distinct regions.

In an embodiment, the linker molecules are printed on the surface of the substrate using non-contact-based printing. In an embodiment, the linker molecules are printed on the surface of the substrate using contact-based printing.

The present application also includes a method of fabricating an array having compartmentalization comprising:
printing linker molecules on a surface of a substrate in a plurality of distinct regions to form a printed array of linker molecules;
adding physical barriers to the surface of the substrate to form one or more compartments,
wherein the physical barriers are added prior or after the printing linker molecules to form the array having compartmentalization.

In an embodiment, the physical barriers are removable. In an embodiment, the physical barriers are non-removable.

In an embodiment, the physical barriers are added prior to printing linker molecules on a surface of a substrate in a plurality of distinct regions. In an embodiment, the physical barriers are added after printing linker molecules on a surface of a substrate in a plurality of distinct regions.

The present application also includes a method of fabricating an array of linker molecules comprising:
printing the linker molecules on a surface of a substrate in a plurality of distinct regions to form the array of linker molecules, wherein the linker molecules are printed using an organic printing solution.

The arrays of the application are useful in assaying a sample. Accordingly, the present application includes a method of assaying a sample comprising:
a) adding a bioreagent solution comprising one or more bioreagents to the one or more distinct regions of the compartmentalized array of linker molecules as defined in any one of claims 1 to 38 to form compartmentalized array of linker molecules bound to the one or more bioreagents;
b) contacting a sample solution containing an analyte with the compartmentalized array of linker molecules bound to one or more bioreagents; and
c) reading the assay to assay the sample.

In an embodiment, the assay is a multiplexed assay.

The arrays of the application are also useful for imaging.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows a top view of the compartmentalized array of linker molecules alternatingly filled with food dye solution in exemplary embodiments of the application.

FIG. 2 shows four bioreagent spots from a bioassay produced by incubating IgG antibody with a compartmentalized array of printed APTES linker in exemplary embodiments of the application.

FIG. 3A-B shows the results of an exemplary immunoassay, A) shows fluorescently labeled protein analyte; and B) shows a negative control.

FIG. 4A-E shows the blocking step in exemplary embodiments of the application, A) shows a schematic diagram of a method of blocking solution incubation, B) shows a fluorescence microscope image of the antibody microarray generated without the blocking step, C) shows a fluorescence microscope image of an antibody microarray generated with conventional BSA blocking method, D) shows a fluorescence microscope image of an antibody microarray generated with polyethylene glycol (PEG) blocker, and E) shows a fluorescence microscope image of an antibody microarray generated with the blocking step according to an exemplary embodiment of the present application.

FIG. 5 shows a schematic diagram of a prior art method of printing an array of bioreagent spots.

FIG. 6 shows a schematic diagram of a method of producing a bioassay in exemplary embodiments of the application.

FIG. 7 shows a flow diagram of the method of FIG. 6.

FIG. 8 shows a schematic diagram of a printed array of linker molecules in exemplary embodiments of the application.

FIG. 9 shows an expanded view of area A of FIG. 8.

FIG. 10 shows a schematic diagram of an exemplary printed array of linker molecules of FIG. 4 compartmentalized into an array of compartments by a physical barrier.

### DESCRIPTION OF VARIOUS EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this application, the singular forms “a”, “an” and “the” include plural references unless the content clearly dictates otherwise.

The term “distinct regions” as used herein refers to areas on the surface of the substrate, separated from each other and defined by a boundary having a predetermined shape and size.

The term “linker molecule” as used herein refers to a moiety that covalently or non-covalently binds a compound of interest.

The term “array” as used herein refers to an arrangement of linker molecules or molecules of interest on a substrate surface.

The term “physical barriers” as used herein refers to barriers which form compartments of sufficiently large dimensions to prevent fluid flow between samples within compartments formed by said barriers. For example, a physically uninterrupted surface may be one which has physical barriers such as walls surrounding the array, which extend above the substrate surface more than 10 micrometers (or more than 5, 2, or 1 micrometers).

The term “non-contact-based printing” as used herein refers to a printing technique where a printing solution or ink is deposited onto a substrate without coming into contact with the substrate.

The term “contact-based printing” as used herein refers to a printing technique where a printing solution or ink is deposited onto a substrate by contacting with the substrate.

The term “micro-contact printing (pCP)” as used herein refers to a soft lithography method, in which an ink solution composed of molecules of interest is transferred from an elastomeric mold, or stamp, to a substrate surface.

The term “pin spotting” as used herein refers to a method of contact-based printing, in which a printing solution or ink composed of molecules of interest is delivered onto the substrate through one or more spotting pins installed on a spotting device.

The term “inkjet printing” as used herein refers to an ejection, from a nozzle, of liquid droplets (ink) composed of molecules of interest which travel a short distance (1-5 mm) through the air to land on a substrate in a pattern.

The term “background regions” as used herein refers to areas between the plurality of distinct regions of linker molecules with minimal or no linker molecules coupled to the substrate.

The term “multiplexed assay” as used herein refers to an assay simultaneously measuring multiple analytes in a single experiment.

The term “array of the application” as used herein refers to the compartmentalized array of printed linker molecules as described herein.

II. Array

The present application includes a compartmentalized array of linker molecules comprising:
a) linker molecules printed on a surface of a substrate in a plurality of distinct regions to form a printed array of linker molecules;
b) physical barriers on the surface, the physical barriers forming one or more compartments surrounding and separating at least a portion of the plurality of distinct regions.

Substrates which can be used in the array of the present application can be fabricated from various materials and are within the consideration of the person skilled in the art. Examples of suitable substrates include, but are not limited to, silicon, glass, rigid plastics, e.g. polytetrafluoroethylene, polypropylene, polystyrene, polycarbonate, and blends thereof, and mica. In some embodiments, the substrate is selected from glass, silicon, polystyrene, and mica.

The substrates can take a variety of configurations. This includes slide or plate configuration, such as a rectangular, square or disc configuration and may vary as required. The size and the shape of the substrate is within the consideration of the person skilled in the art and the particular use of the array.

The surface of the substrate can be smooth or substantially planar, or have periodic irregularities, i.e. a series of depressions forming wells, such as depressions or elevations, or have a porous surface, such as is found in porous glass or silica.

The linker molecules of the present application can be printed on the surface of the substrate using contact-based printing or noncontact-based printing. In some embodiments, the linker molecules are printed on the surface of the substrate using contact-based printing. In some embodiments, the contact-based printing is selected from micro-contact printing and pin spotting.

In some embodiments, the linker molecules are printed on the surface of the substrate using noncontact-based printing. In some embodiments, the noncontact-based printing is inkjet printing.

In some embodiments, the linker molecules are loaded to an inkjet nozzle of an inkjet spotter for printing. In some embodiments, the inkjet spotter is an automated inkjet spotter. In some embodiments, the automated inkjet spotter facilitates the creation of various patterns of the plurality of distinct regions of the linker molecules, as the spotting pattern (spacing, rows, columns, etc.) is defined using the software that controls the spotter. In some embodiments, the volume of each distinct region of the linker molecules is controlled by adjusting the number of droplets delivered from the nozzle using the software. Substrates with desired hydrophilicity/hydrophobicity can also help control the size of each distinct region. In some embodiments, hydrophobic substrates create smaller spots than those on hydrophilic substrates.

The compartmentalized arrays of linker molecules of the present application are selected from microarray and nanoarray. In some embodiments, the compartmentalized arrays of linker molecules are microarrays. In some embodiments, the compartmentalized arrays of linker molecules are nanoarrays.

In some embodiments, the surface of the substrate comprises background regions between the plurality of distinct regions of linker molecules, and wherein the background regions are free from the linker molecules. It is to be understood that the background regions can include regions substantially free, having minimal or no linker molecules coupled to the substrate.

The size and the shape of the distinct regions of the present application can vary according to particular use of the application and is within the consideration of the person skilled in the art. In some embodiments, the plurality of distinct regions of linker molecules are spots. In some embodiments, the spots have a circular or a semi-circular shape.

In some embodiments, the plurality of distinct regions of linker molecules are spots having a diameter greater than 20 nm, 40 nm, 50 nm, 80 nm or 100 nm. In some embodiments, the plurality of distinct regions of linker molecules are spots having a diameter greater than 50 nm.

In some embodiments, the plurality of distinct regions of linker molecules are spots having a diameter greater than 500 nm, 1 micron or 1.5 micron. In some embodiments, the plurality of distinct regions of linker molecules are spots having a diameter greater than 1 micron.

In some embodiments, the linker molecules are selected from an aminosilane, a poly-L-lysine, an epoxysilane, an aldehyde silane, a trichloro(alkyl) silane, and streptavidin. In some embodiments, the aminosilane is ((3-aminopropyl)triethoxysilane). In some embodiments, the epoxysilane is 3-glycidoxypropyldimethoxymethylsilane (GPS).

In some embodiments, the linker molecules are printed on the surface of the substrate using a printing solution. In some embodiments, the printing solution comprises a printing buffer and one or more additives.

In some embodiments, the linker molecules are present in the printing solution in an amount of about 0.5% w/v to about 5% w/v. In some embodiments, the linker molecules are present in the printing solution in an amount of about 1% w/v.

In some embodiments, the printing solution is an aqueous printing solution. In some embodiments, the aqueous printing solution comprises a phosphate-buffered saline (PBS) printing buffer.

In some embodiments, the printing solution is an organic printing solution. In some embodiments, the organic printing solution comprises a dimethyl sulfoxide (DMSO) buffer.

In some embodiments, the printing solution comprises a preservative to increase the shelf-life of the printed array of linker molecules.

In some embodiments, the linker molecules are present in the printing solution in an amount of about 0.5% w/v to about 5% w/v. In some embodiments, the linker molecules are present in the printing solution in an amount of about 1% w/v.

In some embodiments, the printing solution further comprises a glycerol. In some embodiments, the glycerol is present in an amount of about 5% w/v to about 30% w/v. In some embodiments, the glycerol is present in an amount of about 10% w/v to about 20% w/v. In some embodiments, the glycerol is present in an amount of about 15% w/v. In some embodiments, the glycerol prevents evaporation of the solution.

In some embodiments, the one or more additives are selected from trehalose, Tween-20, agarose and alginate. In some embodiments, the one or more additives improve the spatial resolution of the printed array of linker molecules. High spatial resolution can be obtained for example, by reducing the size of the distinct regions of linker molecules printed on the surface of the substrate. The size of the distinct regions of linker molecules can be reduced, for example, by increasing the viscosity of the printing solution. It is to be understood that the higher the viscosity of the printing solution, the higher the spatial resolution of the printed array of linker molecules.

In some embodiments, the printed array of linker molecules is sealed under airtight conditions with desiccant and stored at either room temperature, 4° C. or −20° C.

In some embodiments, the physical barriers used in the present application can be any barrier means of sufficiently large dimensions to prevent mixing of the different samples on the surface of the array. In some embodiments, the physical barriers are structures, separate or interconnected therebetween forming walls between the distinct regions on the surface of the substrate.

In some embodiments, the physical barriers are removable. In some embodiments, the physical barriers are non-removable. It is understood that removable barriers can be added before or after the linker molecules are printed on the surface of the substrate and can be removed according to the use of the array. The non-removable physical barriers refer to physical barriers, fixed to the array and can be assembled by any method known in the art.

In some embodiments, the physical barriers are formed using photolithography. In some embodiments, the physical barriers are formed using a 3D printed polymer. In some embodiments, the polymer is acrylamide or polyethylene glycol (PEG)-based photopolymer.

In some embodiments, the one or more compartments are configured to contain about 1 to about 10 microlitres of a solution. In some embodiments, the one or more compartments are configured to contain about 2 to about 3 microlitres of the solution.

In some embodiments, the array comprises about 75 to about 250 compartments. In some embodiments, the array comprises 80, 100, 120, 150, 170 or 200 compartments.

In some embodiments, the array comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of the plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

III. Methods

(i) Arrays Having Compartmentalization

The present application includes a method of fabricating an array having compartmentalization comprising:
printing linker molecules on a surface of a substrate in a plurality of distinct regions to form a printed array of linker molecules;
adding physical barriers to the surface of the substrate to form one or more compartments,
wherein the physical barriers are added prior or after the printing linker molecules to form the array having compartmentalization.

In some embodiments, the method of fabricating an array having compartmentalization further comprises adding one or more bioreagents to the one or more distinct regions. In some embodiments, the one or more bioreagents are added for incubation with the printed array of linker molecules.

The bioreagent that can be used in the methods of the present application is any bioreagent known in the art. In some embodiments, the one or more bioreagents are selected from antibodies, proteins, nucleic acids, cells, extracellular vesicles (EVs) and exosomes.

In some embodiments, the one or more bioreagents are comprised in a bioreagent solution.

In some embodiments, the one or more bioreagents are bound onto the substrate via the linker molecules by covalent bonds. In some embodiments, the one or more bioreagents are bound onto the substrate via the linker molecules by non-covalent bonds.

In some embodiments, the bioreagent solution comprises a printing buffer and one or more additives. In some embodiments, the printing buffer is a phosphate-buffered saline (PBS). In some embodiments, the printing buffer is a dimethyl sulfoxide (DMSO).

In some embodiments, the bioreagent solution further comprises glycerol. In some embodiments, the glycerol is present in an amount of about 5% w/v to about 30% w/v. In some embodiments, the glycerol is present in an amount of about 10% w/v to about 20% w/v. In some embodiments, the glycerol is present in an amount of about 15% w/v.

In some embodiments, the bioreagent solution is added to the one or more distinct regions under a humidified environment.

In some embodiments, the method of fabricating an array having compartmentalization further comprises adding a blocking solution to the array having compartmentalization. In some embodiments, the blocking solution is co-incubated with the printed linker molecules. In some embodiments, the blocking solution is co-incubated with the printed linker molecules for about 30 seconds to about 5 minutes. In some embodiments, the blocking solution is co-incubated with the printed linker molecules for about 1 minute to about 2 minutes. In some embodiments, the blocking solution is co-incubated with the printed linker molecules for 1 minute, 1.5 minutes, 2 minutes, 2.5 minutes or 3 minutes.

In some embodiments, the blocking solution comprises a non-specific protein blocker and a buffer. In some embodiments, the non-specific protein blocker is bovine serum albumin (BSA). In some embodiments, the buffer is PBS buffer.

In some embodiments, the BSA is present in the blocking solution in an amount of about 0.2% w/v to about 20% w/v. In some embodiments, the BSA is present in an amount of about 0.5% w/v to about 15% w/v. In some embodiments, the BSA is present in an amount of about 10% w/v. In some embodiments, the BSA is present in an amount of about 1% w/v.

In some embodiments, the substrate includes a layer of blocked linker molecules, and the method of fabricating an array having compartmentalization comprises printing an unblocking reagent in the plurality of distinct regions to convert the blocked linker molecules into linker molecules in the plurality of distinct regions. The blocked linker molecules comprise linker molecules bound to a blocking reagent. The unblocking reagent converts the blocked linker molecules into unblocked linker molecules by breaking a connection between the linker molecules and the blocking reagent.

In some embodiments, the blocking reagent is a non-specific protein blocker. In some embodiments, the non-specific protein blocker is BSA. In some embodiments, the unblocking reagent is a detergent solution, a solution with varied pH levels, or a solution having a high salt concentration. In some embodiments, the detergent solution comprises Triton™ X-100. In some embodiments, the method of fabricating an array having compartmentalization further comprises washing the substrate after printing the unblocking reagent in the plurality of distinct regions.

Substrates which can be used in the array of the present application can be fabricated from various materials and are within the consideration of the person skilled in the art. Examples of suitable substrates include, but are not limited to, silicon, glass, rigid plastics, e.g. polytetrafluoroethylene, polypropylene, polystyrene, polycarbonate, and blends thereof, and mica. In some embodiments, the substrate is selected from glass, silicon, polystyrene, and mica.

The substrates can take a variety of configurations. This includes slide or plate configuration, such as a rectangular, square or disc configuration and may vary as required. The size and the shape of the substrate is within the consideration of the person skilled in the art and the particular use of the array.

The surface of the substrate can be smooth or substantially planar, or have periodic irregularities, i.e. a series of depressions forming wells, such as depressions or elevations, or have a porous surface, such as is found in porous glass or silica.

The linker molecules of the present application can be printed on the surface of the substrate using contact-based printing or noncontact-based printing. In some embodiments, the linker molecules are printed on the surface of the substrate using contact-based printing. In some embodiments, the contact-based printing is selected from micro-contact printing and pin spotting.

In some embodiments, the linker molecules are printed on the surface of the substrate using noncontact-based printing. In some embodiments, the noncontact-based printing is inkjet printing.

In some embodiments, the linker molecules are loaded to an inkjet nozzle of an inkjet spotter for printing. In some embodiments, the inkjet spotter is an automated inkjet spotter. In some embodiments, the automated inkjet spotter facilitates the creation of various patterns of the plurality of distinct regions of the linker molecules, as the spotting pattern (spacing, rows, columns, etc.) is defined using the software that controls the spotter. In some embodiments, the volume of each distinct region of the linker molecules is controlled by adjusting the number of droplets delivered from the nozzle using the software. Substrates with desired hydrophilicity/hydrophobicity can also help control the size of each distinct region. In some embodiments, hydrophobic substrates create smaller spots than those on hydrophilic substrates.

The compartmentalized array of linker molecules of the present application is selected from microarray and nanoarray. In some embodiments, the compartmentalized array of linker molecules is a microarray. In some embodiments, the compartmentalized array of linker molecules is a nanoarray.

In some embodiments, the surface of the substrate comprises background regions between the plurality of distinct regions of linker molecules, and wherein the background regions are free from the linker molecules. It is to be understood that the background regions can include regions substantially free, having minimal or no linker molecules coupled to the substrate.

The size and the shape of the distinct regions of the present application can vary according to particular use of the application and is within the consideration of the person skilled in the art. In some embodiments, the plurality of distinct regions of linker molecules are spots. In some embodiments, the spots have a circular or a semi-circular shape.

In some embodiments, the plurality of distinct regions of linker molecules are spots having a diameter greater than 20 nm, 40 nm, 50 nm, 80 nm or 100 nm. In some embodiments, the plurality of distinct regions of linker molecules are spots having a diameter greater than 50 nm.

In some embodiments, the plurality of distinct regions of linker molecules are spots having a diameter greater than 500 nm, 1 micron or 1.5 micron. In some embodiments, the plurality of distinct regions of linker molecules are spots having a diameter greater than 1 micron.

In some embodiments, the linker molecules are selected from an aminosilane, a poly-L-lysine, an epoxysilane, an aldehyde silane, a trichloro(alkyl) silane, and streptavidin. In some embodiments, the aminosilane is ((3-aminopropyl)triethoxysilane). In some embodiments, the epoxysilane is 3-glycidoxypropyldimethoxymethylsilane (GPS).

In some embodiments, the linker molecules are printed on the surface of the substrate using a printing solution. In some embodiments, the printing solution comprises a printing buffer and one or more additives.

In some embodiments, the linker molecules are present in the printing solution in an amount of about 0.5% w/v to about 5% w/v. In some embodiments, the linker molecules are present in the printing solution in an amount of about 1% w/v.

In some embodiments, the printing solution is an aqueous printing solution. In some embodiments, the aqueous printing solution comprises a PBS printing buffer.

In some embodiments, the printing solution is an organic printing solution. In some embodiments, the organic printing solution comprises a DMSO buffer.

In some embodiments, the printing solution comprises a preservative to increase the shelf-life of the printed array of linker molecules.

In some embodiments, the printing solution further comprises glycerol. In some embodiments, the glycerol is present in an amount of about 5% w/v to about 30% w/v. In some embodiments, the glycerol is present in an amount of about 10% w/v to about 20% w/v. In some embodiments, the glycerol is present in an amount of about 15% w/v. In some embodiments, the glycerol prevents evaporation of the solution.

In some embodiments, the one or more additives are selected from trehalose, Tween-20, agarose and alginate. In some embodiments, the one or more additives improve the spatial resolution of the printed array of linker molecules. High spatial resolution can be obtained for example, by reducing the size of the distinct regions of linker molecules printed on the surface of the substrate. The size of the distinct regions of linker molecules can be reduced, for example, by increasing the viscosity of the printing solution. It is to be understood that the higher the viscosity of the printing solution, the higher the spatial resolution of the printed array of linker molecules. In some embodiments, the printing solution does not come into direct contact with the bioreagent solution, reducing or eliminating the interactions between the one or more additives and the bioreagent solution.

In some embodiments, the physical barriers used in the present application can be any barrier means of sufficiently large dimensions to prevent mixing of the different samples on the surface of the array. In some embodiments, the physical barriers are structures, separate or interconnected therebetween forming walls between the distinct regions on the surface of the substrate.

In some embodiments, the physical barriers are removable. In some embodiments, the physical barriers are non-removable. It is understood that removable barriers can be added before or after the linker molecules are printed on the surface of the substrate and can be removed according to the use of the array. The non-removable physical barriers refer to physical barriers, fixed to the array and can be assembled by any method known in the art.

In some embodiments, the physical barriers are formed using photolithography. In some embodiments, the physical barriers are formed using a 3D printed polymer. In some embodiments, the polymer is acrylamide or PEG-based photopolymer.

In some embodiments, the method of fabricating an array having compartmentalization comprises first printing linker molecules on a surface of a substrate in a plurality of distinct regions to form a printed array of linker molecules and then adding physical barriers to the surface of the substrate to form one or more compartments. In some embodiments, the physical barriers added to the surface of the substrate surround and separate at least a portion of the plurality of distinct regions.

In some embodiments, when the physical barriers are formed using photolithography, the physical barriers are added prior to printing linker molecules on a surface of a substrate in a plurality of distinct regions. It is to be understood that the linker molecules are printed in a plurality of distinct regions within the one or more compartments formed by the physical barriers such that the plurality of distinct regions are separated and surrounded by the physical barriers. In said embodiments, the physical barriers are non-removable.

In some embodiments, the physical barriers are added after printing linker molecules on a surface of a substrate in a plurality of distinct regions.

In some embodiments, the one or more compartments are configured to contain about 1 to about 10 microlitres of a solution. In some embodiments, the one or more compartments are configured to contain about 2 to about 3 microlitres of the solution.

In some embodiments, the array comprises about 75 to about 250 compartments. In some embodiments, the array comprises 80, 100, 120, 150, 170 or 200 compartments.

In some embodiments, the array comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of the plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

(ii) Array Printed Using an Organic Printing Solution

The present application also includes a method of fabricating an array of linker molecules comprising:
printing the linker molecules on a surface of a substrate in a plurality of distinct regions to form the array of linker molecules, wherein the linker molecules are printed using an organic printing solution.

In some embodiments, the organic printing solution comprises a printing solvent and one or more additives. In some embodiments, the printing solvent is DMSO.

In some embodiments, the printing solution comprises a preservative to increase the shelf-life of the printed array of linker molecules.

In some embodiments, the organic printing solution further comprises glycerol. In some embodiments, the glycerol is present in an amount of about 5% w/v to about 30% w/v. In some embodiments, the glycerol is present in an amount of about 10% w/v to about 20% w/v. In some embodiments, the glycerol is present in an amount of about 15% w/v. In some embodiments, the glycerol prevents evaporation of the solution.

In some embodiments, the one or more additives are selected from trehalose, Tween-20, agarose and alginate. In some embodiments, the one or more additives improve the spatial resolution of the printed array of linker molecules. High spatial resolution can be obtained for example, by reducing the size of the distinct regions of linker molecules printed on the surface of the substrate. The size of the distinct regions of linker molecules can be reduced, for example, by increasing the viscosity of the printing solution. It is to be understood that the higher the viscosity of the printing solution, the higher the spatial resolution of the printed array of linker molecules.

The linker molecules of the present application can be printed on the surface of the substrate using contact-based printing or noncontact-based printing. In some embodiments, the linker molecules are printed on the surface of the substrate using contact-based printing. In some embodiments, the contact-based printing is selected from micro-contact printing and pin spotting.

In some embodiments, the linker molecules are printed on the surface of the substrate using noncontact-based printing. In some embodiments, the noncontact-based printing is inkjet printing.

In some embodiments, the linker molecules are loaded to an inkjet nozzle of an inkjet spotter for printing. In some embodiments, the inkjet spotter is an automated inkjet spotter. In some embodiments, the automated inkjet spotter facilitates the creation of various patterns of the plurality of distinct regions of the linker molecules, as the spotting pattern (spacing, rows, columns, etc.) is defined using the software that controls the spotter. In some embodiments, the volume of each distinct region of the linker molecules is controlled by adjusting the number of droplets delivered from the nozzle using the software. Substrates with desired hydrophilicity/hydrophobicity can also help control the size of each distinct region. In some embodiments, hydrophobic substrates create smaller spots than those on hydrophilic substrates.

The size and the shape of the distinct regions of the present application can vary according to particular use of the application and is within the consideration of the person skilled in the art. In some embodiments, the plurality of distinct regions of linker molecules are spots. In some embodiments, the spots have a circular or a semi-circular shape.

In some embodiments, the plurality of distinct regions of linker molecules are spots having a diameter greater than 20 nm, 40 nm, 50 nm, 80 nm or 100 nm. In some embodiments, the plurality of distinct regions of linker molecules are spots having a diameter greater than 50 nm.

In some embodiments, the plurality of distinct regions of linker molecules are spots having a diameter greater than 500 nm, 1 micron or 1.5 micron. In some embodiments, the plurality of distinct regions of linker molecules are spots having a diameter greater than 1 micron.

In some embodiments, the linker molecules are selected from an aminosilane, a poly-L-lysine, an epoxysilane, an aldehyde silane, a trichloro(alkyl) silane, and streptavidin. In some embodiments, the aminosilane is ((3-aminopropyl)triethoxysilane). In some embodiments, the epoxysilane is 3-glycidoxypropyldimethoxymethylsilane (GPS).

In some embodiments, the linker molecules are present in the printing solution in an amount of about 0.5% w/v to about 5% w/v. In some embodiments, the linker molecules are present in the printing solution in an amount of about 1% w/v.

In some embodiments, the method further comprises treating the substrate with a blocking solution after the printing.

In some embodiments, the blocking solution comprises a non-specific protein blocker and a buffer. In some embodiments, the non-specific protein blocker is BSA. In some embodiments, the buffer is PBS.

In some embodiments, the BSA is present in the blocking solution in an amount of about 0.2% w/v to about 20% w/v. In some embodiments, the BSA is present in an amount of about 0.5% w/v to about 15% w/v. In some embodiments, the BSA is present in an amount of about 10% w/v. In some embodiments, the BSA is present in an amount of about 1% w/v.

In some embodiments, the array comprises at least two compartments as described above.

Substrates which can be used in the array of the present application can be fabricated from various materials and are within the consideration of the person skilled in the art. Examples of suitable substrates include, but are not limited to, silicon, glass, rigid plastics, e.g. polytetrafluoroethylene, polypropylene, polystyrene, polycarbonate, and blends thereof, and mica. In some embodiments, the substrate is selected from glass, silicon, polystyrene, and mica.

In some embodiments, an array is fabricated by the method of fabricating an array having compartmentalization as described above.

IV. Methods of Using of the Arrays of the Application

The present application includes a use of the compartmentalized arrays of linker molecules of the present application for assaying a sample.

The present application also includes a method of assaying a sample comprising:
a) adding a bioreagent solution comprising one or more bioreagents to the one or more distinct regions of the compartmentalized array of linker molecules of the present application to form compartmentalized array of linker molecules bound to the one or more bioreagents;
b) contacting a sample solution containing an analyte with the compartmentalized array of linker molecules bound to one or more bioreagents; and
c) reading the assay to assay the sample.

The bioreagent that can be used in the methods of the present application is any bioreagent known in the art. In some embodiments, the one or more bioreagents are selected from antibodies, proteins, nucleic acids, cells, extracellular vesicles (EVs) and exosomes.

In some embodiments, the one or more bioreagents are bound onto the substrate via the linker molecules by covalent bonds. In some embodiments, the one or more bioreagents are bound onto the substrate via the linker molecules by non-covalent bonds. In some embodiments, the one or more bioreagents are added for incubation with the printed array of linker molecules.

In some embodiments, the bioreagent solution comprises a printing buffer and one or more additives. In some embodiments, the printing buffer is a phosphate-buffered saline (PBS). In some embodiments, the printing buffer is a dimethyl sulfoxide (DMSO).

In some embodiments, the bioreagent solution further comprises glycerol. In some embodiments, the glycerol is present in an amount of about 5% w/v to about 30% w/v. In some embodiments, the glycerol is present in an amount of about 10% w/v to about 20% w/v. In some embodiments, the glycerol is present in an amount of about 15% w/v.

In some embodiments, the bioreagent solution is added to the one or more distinct regions under a humidified environment.

In some embodiments, the methods of using the arrays of the present application further comprise adding a blocking solution to the array. In some embodiments, the blocking solution is co-incubated with the printed linker molecules. In some embodiments, the blocking solution is co-incubated with the printed linker molecules for about 30 seconds to about 5 minutes. In some embodiments, the blocking solution is co-incubated with the printed linker molecules for about 1 minute to about 2 minutes. In some embodiments, the blocking solution is co-incubated with the printed linker molecules for 1 minute, 1.5 minutes, 2 minutes, 2.5 minutes or 3 minutes.

In some embodiments, the blocking solution comprises a non-specific protein blocker and a buffer. In some embodiments, the non-specific protein blocker is bovine serum albumin (BSA). In some embodiments, the buffer is PBS buffer.

In some embodiments, the BSA is present in the blocking solution in an amount of about 0.2% w/v to about 20% w/v. In some embodiments, the BSA is present in an amount of about 0.5% w/v to about 15% w/v. In some embodiments, the BSA is present in an amount of about 10% w/v. In some embodiments, the BSA is present in an amount of about 1% w/v.

In some embodiments, the physical barriers are removed prior to reading the assay.

In some embodiments, the assay is a multiplexed assay. In some embodiments, the multiplexed assay is performed for simultaneous detection of multiple analytes.

In some embodiments, the sample solution containing the analyte is selected from cell media, blood, urine, saliva, mucus, and other complex biofluids.

Analytes that can be detected in the assay according to the method of the present application are any analytes known in the art. Examples of suitable analytes include but are not limited to antigens, antibodies, nucleic acids, proteins, small molecules, hormones, receptors, ligands, extracellular vesicles (EVs), exosomes and the like. In some embodiments, the analyte is selected from antibodies, proteins, nucleic acids, cells, extracellular vesicles (EVs), exosomes, and combinations thereof. In some embodiments, the analyte is the antibody.

The assay that can be conducted using the array and methods of the present application are any bioassays known in the art. Examples of assay include but are not limited to immunoassay, proteomic assay and the like. In some embodiments, the assay is immunoassay. It is understood that any assay conducted for diagnostic, drug screening or research can be conducted using the array and methods of the present application.

In some embodiments, the assay can be read using any suitable detection apparatus such as fluorometer, spectrometer, camera and the like. In some embodiments, the detection method utilized by the detection apparatus is selected from laser induced luminescence, FRET (fluorescence resonance energy transfer), fluorescence polarization, transmittance, fluorescence anisotropy, raman spectroscopy or color change.

The present application also includes a use of an array of the present application for imaging. In some embodiments, imaging includes using the array of the present application with microscopes, environment-control chamber for cell analysis, surface plasmon resonance spectrometers.

The following non-limiting examples are illustrative of the present application.

### EXAMPLES

General Methods and Materials

### Example 1: Preparation of Printed Array of Linker Molecules Bound to Bioreagents

To form the (3-Aminopropyl)triethoxysilane (APTES) linker array, 1% w/v of aminosilane linker (purchased from Sigma-Aldrich) was printed on cleaned glass with a pin microarray spotter (μArrayer from LabNEXT) in a printing solution of PBS buffer with 15% w/v glycerol (to prevent drying). After incubating in a humidified chamber for 1 hour, excessive linker molecules were washed off, followed by incubating with a 3 uL antibody solution (commercial fluorescein isothiocyanate (FITC) labeled rabbit IgGs at 10 ug/mL in PBS) for 1 h and then washing to form the antibody microarray.

### Example 2: Inkjet Printing

Linker molecules are diluted in water (e.g., APTES) or DMSO (e.g. GPS) with 15% glycerol, and loaded to an inkjet nozzle for printing. Glycerol helps to prevent evaporation.

### Example 3: Preparation of Array of Linker Molecules by Using Organic Printing Solution

1% w/v 3-glycidoxypropyldimethoxymethylsilane (GPS, ordered from Sigma-Aldrich) was prepared by diluting it in DMSO and printed on a cleaned glass slide. After 20 minute incubation, the glass slide was rinsed with fresh DMSO to remove unbound GPS molecules. The slide was then dried and stored in an oven for 30 minutes at 120° C., followed by ultrasonication in fresh toluene for 15 minutes. The slide was then rinsed with fresh ethanol and dried.

### Example 4: Compartmentalized Array of Linker Molecules

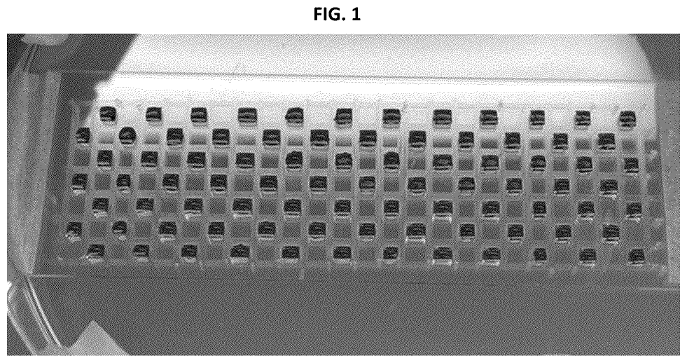

The linker molecules were printed on the surface of the substrate in a plurality of distinct regions according to the method of the present application. Physical barriers were added to the surface of the substrate. The compartments of the array were alternatively filled with food dye, to demonstrate the compartmentalization for cross-contamination-free multiplexed assays (FIG. 1). Alternatively, physical barriers were added to the surface of the substrate first, then the linker molecules were printed on the surface into each compartment to form compartmentalized linker arrays.

### Example 5: Bioassay

Nanoliter linker spots of APTES and GPS linkers were printed using a pin microarray spotter without evaporation, by adding 15% glycerol and using aqueous and the organic printing solutions, namely the nonvolatile PBS and DMSO as the printing buffer, respectively, and achieved satisfactory results in both experiments with antibody binding signal intensity similar to conventional continuous-layer linker approach. Antibody binding signals were not detected from background areas after 1-hour incubation.

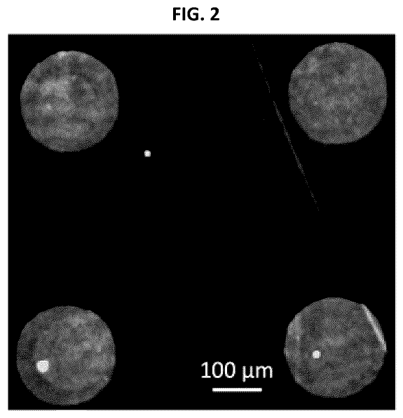

2 μL of IgG antibody bioreagent solution was incubated with a printed APTES linker array in a compartment, demonstrating the consistent spot morphology and low background noise. The antibodies were fluorescently labeled. The image was obtained from a fluorescence microscope (Nikon Eclipse Ti) (FIG. 2).

### Example 6: Immunoassay

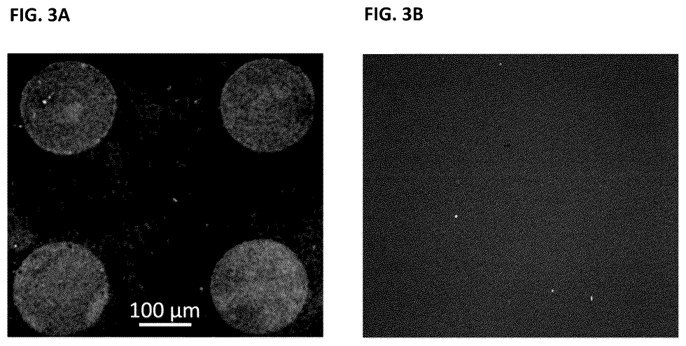

To conduct an immunoassay, the antibody microarray prepared according to procedure of example 5 was incubated with 1% w/v BSA for blocking, then with fluorescently labeled protein analyte (FIG. 3A) or PBS as a negative control (FIG. 3B). A buffer solution spiked with rabbit IgG was used as a test solution. The fluorescent signals obtained from the specific target of interest was higher than that of the negative control, demonstrating the usability of the linker array for highly specific protein detection.

### Blocking Step

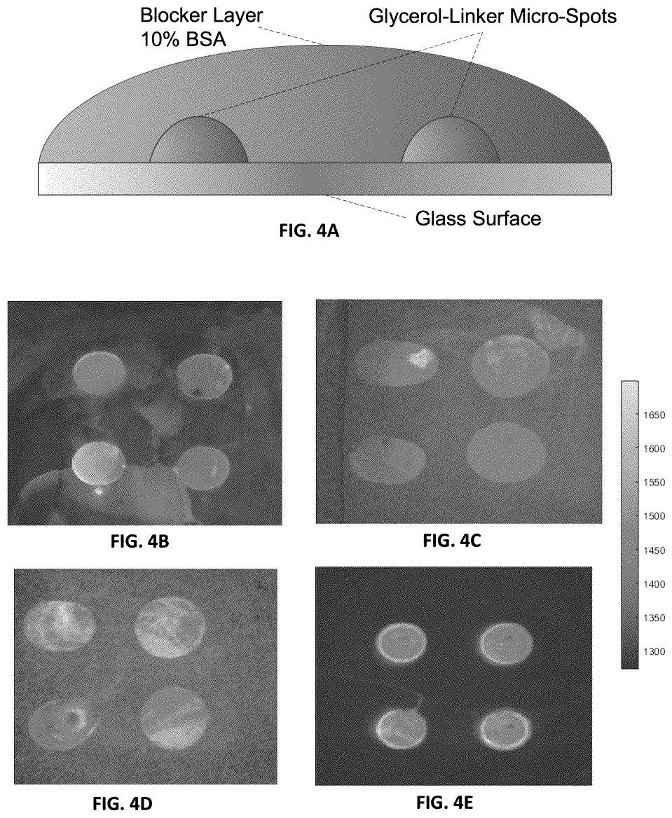

It was noticed that some antibodies surprisingly tend to attach to the glass background even without any linker molecules, therefore, new blocking approach to de-activate the glass background has been developed. 1% w/v APTES linker molecules were printed with a pin spotter on glass in PBS buffer with 15% glycerol and incubated in a humidified chamber for 1 hour. After that, a blocking solution containing 10% BSA in PBS was applied onto the glass and co-incubated with the linker/glycerol droplets for 1-2 minutes (FIG. 4A).

As glycerol is heavier than water, it forms a “two-phase” system that helps maintain the linker spot morphology. The short BSA incubation duration helped limit the diffusion of the BSA molecules into the bottom of the linker spots. Then, the glass slide was washed with PBS, dried, and stored. To prepare the FITC labeled rabbit IgG antibody microarray, the printed array of linker molecules was incubated with an antibody solution for 1 hour and washed (FIG. 4E). FIG. 4B is a fluorescence microscope image of the antibody microarray generated without a blocking step, showing the antibodies randomly attached to the glass background even after washing. This demonstrated the need of a blocking step to de-activate the glass background. FIG. 4C is a fluorescence microscope image of the antibody microarray generated with conventional BSA blocking method, i.e. after the linker array was spotted, washed, and dried, a BSA solution was applied. It showed that the BSA molecules interacted with the linkers, changed the morphology of the linker spots, and occupied some linker areas, resulting in lower antibody binding onto the linkers. FIG. 4D showed the blocking results with polyethylene glycol (PEG). It had similar issues with BSA, and the blocking of the glass background was inefficient. FIG. 4E is an image of the antibody microarray fabricated with the blocking procedure according to the present application. All the spots were well confined, and the antibody binding onto the linker spots were efficient, with clear background, demonstrating the success of this approach.

### Results and Discussion

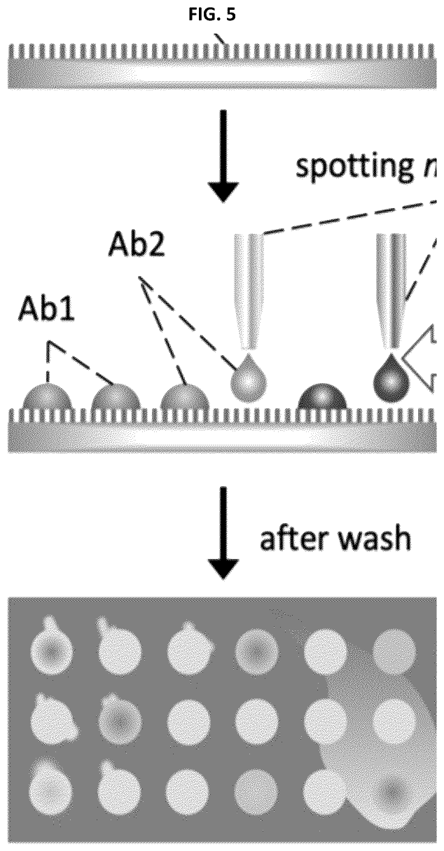

In contrast to the methods of the present disclosure, for conventional bioreagent arrays, bioreagent spots are printed onto a substrate pre-coated with a continuous layer of linker molecules (FIG. 5). Each bioreagent spot is printed as a separate droplet of solution containing a predetermined bioreagent, with the size and shape of each bioreagent spot depending on the size, shape, and physical properties of its corresponding bioreagent droplet. Bioreagent within each droplet reacts with the chemical linker molecule on the substrate to couple the bioreagent to the substrate, resulting in a spot of bioreagent having a size and a shape at least approximately corresponding to the size and the shape of the bioreagent droplet. As such, the presence of the continuous layer of chemical linker molecules causes any variations in bioreagent droplet properties to also result in variations in spot properties, thereby reducing the sensitivity and the reproducibility of the assay. Additionally, non-specific binding or tailing may occur during the printing process to further reduce assay quality. De-activation of the continuous substrate layer after droplet deposition may reduce these effects, but typically degrades sensitivity by also causing damage to the sensitive bioreagents. Bioreagents are typically printed in extensively optimized spotting buffers containing additives (e.g. to adjust the viscosity of the buffer, to prevent solution drying during spotting, etc.) which may impair the bioactivity of the bioreagent.

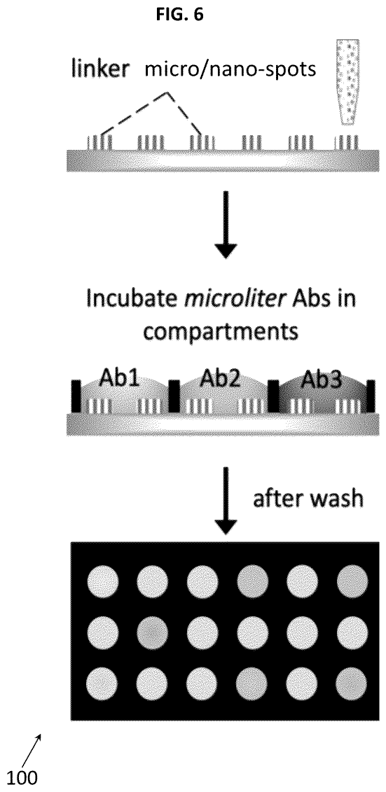

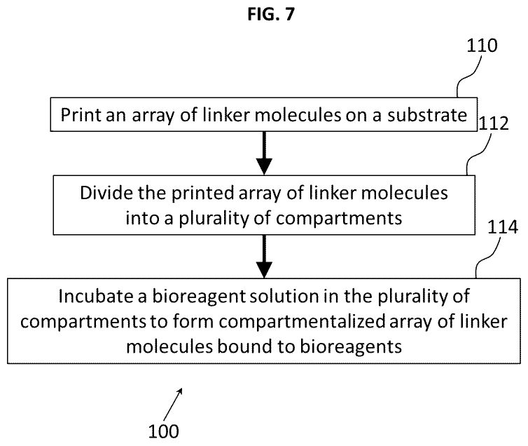

FIG. 6 shows a schematic diagram of a method 100 of producing a bioassay according to exemplary embodiment of the present application. FIG. 7 shows a flow diagram of the method 100. At 110, an array of linker molecules is printed on a surface of the substrate to produce a printed array of linker molecules. At 112, the printed array of linker molecules is divided into a plurality of compartments by adding removable physical barriers. At 114, bioreagent solution is incubated in the plurality of compartments to form compartmentalized array of linker molecules bound to one or more bioreagents. Unlike conventional microtiter plate-based assays, the linker molecules in each compartment serve as technical replicates, which may keep a high multiplexing capacity while improving the assay sensitivity and reproducibility.

In contrast to an array in which the bioreagent is printed, the array of the present application, in which bioreagent is incubated achieves (i) higher assay sensitivity due to preservation of bioreagent activity via incubation in conventional buffers without additives, and lower background noise since after washing, sample analytes will not attach to background regions where no linkers are present; (ii) higher reproducibility due to improved morphology of linker molecules distinct regions (i.e., signals are better confined to the linker distinct regions with reduced tailing effects) and more consistent background signals across the substrate; (iii) improved array accessibility, since the printed array of linker molecules is more robust than an array of bioreagents and is therefore more resistant to degradation upon storage of the linker arrays (e.g. end-users can store substrates having a printed array of linker molecules, and then retrieve substrates as needed to customize their bioassays with their own bioreagents without the need for a complex microarray spotter); or (iv) simpler assay preparation as it mitigates the need to optimize printing buffers for each bioreagent.

Further, in contrast to conventional bioreagent arrays where a mixture of bioreagents is applied, leading to cross-reactions and irreproducible results, the compartmentalization of the printed array of linker molecules of the present application avoids reagent mixing and hence minimizes cross-reactions, which may improve assay reproducibility.

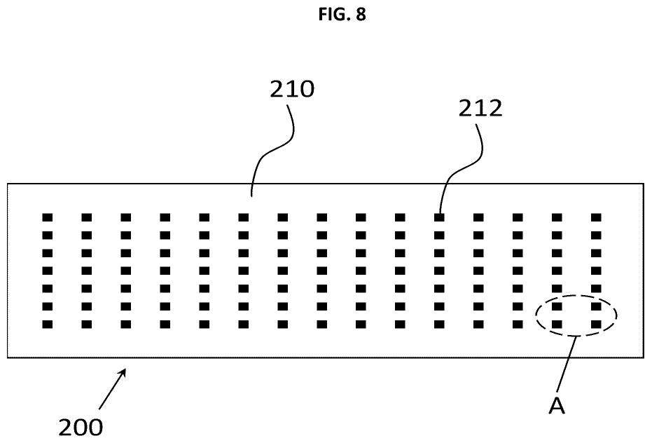

FIG. 8 is a schematic diagram of a printed array of linker molecules 200. The printed array of chemical linker 200 includes a substrate 210 and a plurality of distinct regions 212.

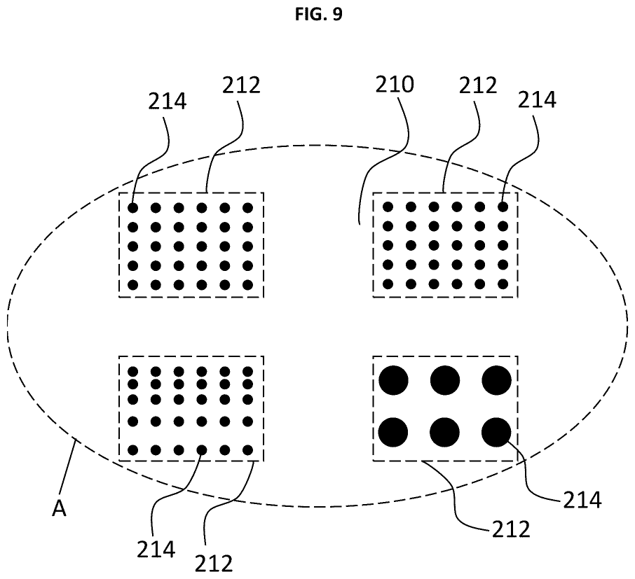

FIG. 9 is an expanded view of area A of FIG. 8. Each distinct region 212 includes a plurality of spots 214, where each spot 214 includes a layer of linker molecules. FIG. 9 shows distinct regions 212, in which the size, position, and pattern of spots vary between the distinct regions 212. One distinct region 212 has a gradient spacing between spots 214. A gradient spacing within the distinct region 212 allows for deposition of a gradient of bioreagent within a sub-array.

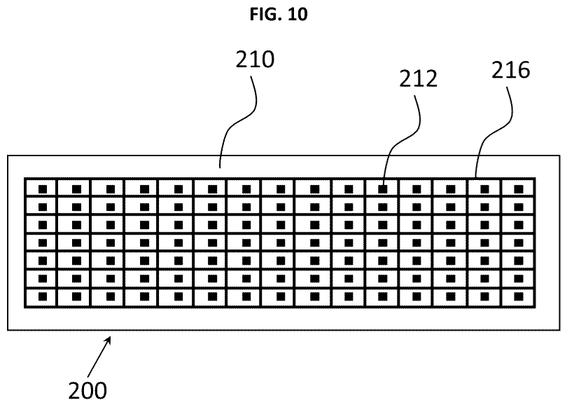

FIG. 10 is a schematic diagram of the printed array of linker molecules 200 compartmentalized into an array of compartments by a physical barrier 216 according to the method of the present application.

The linker molecules were printed using contact-based printing methods and non-contact-based printing methods The use of contact-based printing methods (e.g. micro-contact printing, pin spotting) is associated with capillary forces between the substrate and the printing solution to allow printing to take place, since these forces allow transfer of the printing solution to the substrate. If the substrate is hydrophobic while the printing solution is water-based, contact printing cannot be used. Thus, using non-contact printing techniques (such as ink-jet printing) allows a broader range of substrates to be used.

Further, the use of compartments enables printing linker molecules on unrestricted substantially planar surfaces which increases the scalability of the printing method. In addition, it also enables the use of post-assay data acquisition methods that require flat surfaces, such as optical microscopes.

To conclude, the present application provides printed arrays of linker molecules which may be used in multiplexed bioanalysis, such as bioassays. A printed array of linker molecules subsequently bound to one or more bioreagents via incubation improves at least one of: bioassay sensitivity, bioassay reproducibility, bioassay accessibility, or the simplicity of bioassay preparation relative to a printed array of bioreagent spots.

1. A compartmentalized array of linker molecules comprising:
a) linker molecules printed on a surface of a substrate in a plurality of distinct regions to form a printed array of linker molecules;
b) physical barriers on the surface, the physical barriers forming one or more compartments surrounding and separating at least a portion of the plurality of distinct regions.

2. The compartmentalized array of linker molecules of claim 1, wherein the substrate is selected from glass, silicon, polystyrene, and mica.

3. The compartmentalized array of linker molecules of claim 1, wherein the compartmentalized array of linker molecules is a microarray.

4. The compartmentalized array of linker molecules of claim 1, wherein the surface of the substrate comprises background regions between the plurality of distinct regions of linker molecules, and wherein the background regions are free from the linker molecules.

5. The compartmentalized array of linker molecules of claim 1, wherein the plurality of distinct regions of linker molecules are spots having a diameter greater than 50 nm.

6. The compartmentalized array of linker molecules of claim 1, wherein the physical barriers are removable.

7. The compartmentalized array of linker molecules of claim 1, wherein the one or more compartments are configured to contain about 1 to about 10 microlitres of a solution.

8. The compartmentalized array of linker molecules of claim 1, wherein the array comprises at least two compartments and the plurality of distinct regions in each of the at least two compartments optionally comprise different arrangements of the distinct regions.

9. A method of fabricating an array having compartmentalization comprising:
printing linker molecules on a surface of a substrate in a plurality of distinct regions to form a printed array of linker molecules;
adding physical barriers to the surface of the substrate to form one or more compartments,
wherein the physical barriers are added prior or after the printing linker molecules to form the array having compartmentalization.

10. The method of claim 9, further comprises adding one or more bioreagent to the one or more distinct regions, wherein the one or more bioreagents are selected from antibodies, proteins, nucleic acids, cells, extracellular vesicles (EVs) and exosomes.

11. The method of claim 10, wherein the one or more bioreagents are bound onto the substrate via the linker molecules by covalent bonds.

12. The method of claim 11, further comprises adding a blocking solution to the array having compartmentalization.

13. The method of claim 12, wherein the blocking solution comprises a non-specific protein blocker and a buffer.

14. The method of claim 13, wherein the non-specific protein blocker is bovine serum albumin (BSA) present in the blocking solution in an amount of about 0.2% w/v to about 20% w/v.

15. The method of claim 9, wherein the printing of the linker molecules comprises non-contact-based printing.

16. The method of claim 9, wherein the linker molecules are selected from an aminosilane, a poly-L-lysine, an epoxysilane, an aldehyde silane, a trichloro(alkyl) silane, and streptavidin.

17. The method of claim 9, wherein the linker molecules are printed on the surface of the substrate using a printing solution.

18. The method of claim 17, wherein the printing solution further comprises glycerol present in an amount of about 5% w/v to about 30% w/v.
